(19) United States
(12) Patent Application Publication
(10) Pub. No.: US 2010/0145205 A1
(43) Pub. Date: Jun. 10, 2010

(54) ANALYZING ALTERNANS FROM MEASUREMENTS OF AN AMBULATORY ELECTROCARDIOGRAPHY DEVICE

(75) Inventors: Ali Haghighi-Mood, Andover, MA (US); Lahn Fendelander, Arlington, MA (US); Richard J. Cohen, Chestnut Hill, MA (US)

Correspondence Address: FISH & RICHARDSON P.C., P.O. BOX 1022, MINNEAPOLIS, MN, 55440-1022, US

(73) Assignee: Cambridge Heart, Inc., Bedford, MA (US)

(21) Appl. No.: 12/630,726

(22) Filed: Dec. 3, 2009

Related U.S. Application Data
(60) Provisional application No. 61/120,168, filed on Dec. 5, 2008.

(51) Int. Cl. A61B 5/0452

(52) U.S. Cl. 600/509

(57) ABSTRACT

Cardiac signal data of heart beats measured with an ambulatory electrocardiography device is accessed. The cardiac signal data is segmented into cardiac signal data segments such that each cardiac signal data segment includes cardiac signal data of sequential heart beats. Whether alternans is present in the cardiac signal data segment is determined for each of multiple cardiac signal data segments. Characteristics of the alternans is determined by analyzing cardiac signal data segments for which alternans is determined to be present or by analyzing characteristics of cardiac signal data segments for which alternans is determined to be present.

### CROSS-REFERENCE TO RELATED APPLICATIONS

This application claims priority to U.S. Provisional Application No. 61/120,168, which was filed on Dec. 5, 2008 and titled “ANALYZING ALTERNANS FROM MEASUREMENTS OF AN AMBULATORY ELECTROCARDIOGRAPHY DEVICE,” which is incorporated by reference in its entirety.

### TECHNICAL FIELD

This disclosure is directed to the measurement and processing of data recorded by an ambulatory electrocardiography device.

### BACKGROUND

An ambulatory electrocardiography device is used to measure cardiac electrical signals from a patient, generally outside of a hospital or other medical institution. The device can record signals for extended periods of time (e.g., 24 hours) on a storage medium while the patient goes about a normal daily routine. The patient generally wears the device on his/her person.

### SUMMARY

In general, in some aspects, a method includes accessing cardiac signal data of heart beats measured with an ambulatory electrocardiography device and segmenting the cardiac signal data into cardiac signal data segments, each cardiac signal data segment including cardiac signal data of sequential heart beats. The method also includes determining, for each of multiple cardiac signal data segments, whether alternans is present in the cardiac signal data segment and determining characteristics of the alternans by analyzing cardiac signal data segments, or characteristics of those cardiac signal data segments, for which alternans is determined to be present.

This and other implementations can optionally include one or more of the following features, which also may optionally be in any combination. For example, the method can also include determining, for one or more of the cardiac signal data segments, a heart rate pertaining to the cardiac signal data segment. Determining a heart rate pertaining to the cardiac signal data segment can include determining an average heart rate of the heart beats of the cardiac signal data in the cardiac signal data segment. Determining characteristics of the alternans can include analyzing the determined heart rates pertaining to the cardiac signal data segments for which alternans is determined to be present. Analyzing the determined heart rates pertaining to the cardiac signal data segments for which alternans is determined to be present can include determining an onset heart rate of alternans for the cardiac signal data segments. Analyzing the determined heart rates pertaining to the cardiac signal data segments for which alternans is determined to be present can include determining a maximum heart rate below which alternans is not present for the cardiac signal data segments.

Also, determining characteristics of the alternans can include determining a presence or absence of alternans sustained for a period of time for the cardiac signal data segments. The method can also include measuring the heart beats with the ambulatory electrocardiography device, generating the cardiac signal data with a processor of the ambulatory electrocardiography device, and storing the cardiac signal data in a storage unit of the ambulatory electrocardiography device. Accessing the cardiac signal data of heart beats measured with the ambulatory electrocardiography device can include accessing the stored cardiac signal data in the storage unit with a device other than the ambulatory electrocardiography device. Segmenting the cardiac signal data can include segmenting the cardiac signal data such that the sequential order of the heart beats as measured by the ambulatory electrocardiography device is maintained within the cardiac signal data segments.

Further, segmenting the cardiac signal data can include segmenting the cardiac signal data such that the cardiac signal data in each cardiac signal data segment partially overlaps the cardiac signal data of another cardiac signal data segment. Segmenting the cardiac signal data can include segmenting the cardiac signal data such that each cardiac signal data segment includes cardiac signal data of at least one measured heart beat which is also included in another cardiac signal data segment. Segmenting the cardiac signal data can include segmenting the cardiac signal data such that the cardiac signal data of each measured heart beat is included in at least one cardiac signal data segment along with the cardiac signal data of the sequentially previous measured heart beat and the cardiac signal data of the sequentially following measured heart beat.

Moreover, determining, for each of multiple cardiac signal data segments, whether alternans is present in the cardiac signal data segment can include making a separate determination of whether alternans is present for each of the cardiac signal data segments. Making a separate determination of whether alternans is present for each of the cardiac signal data segments can include conducting spectral or analytic processing separately on each cardiac signal data segment. The method can further include determining that alternans is present in a first cardiac signal data segment and is not present in a second cardiac signal data segment. The method can additionally include rendering information of the determined characteristics of the alternans. Accessing cardiac signal data of heart beats measured with an ambulatory electrocardiography device can include accessing cardiac signal data of heart beats measured with an implanted device. Moreover, the method can include determining the existence of changes in the electrocardiographic waveforms which persist over multiple beats. Determining the existence of changes in the electrocardiographic waveforms can include determining ST segment changes.

The method can also include determining physical activities occurring during measurement of cardiac signal data with the ambulatory electrocardiography device, and analyzing the determined characteristics of the alternans based on the determined physical activities. The method can further include assessing, based at least in part on the determined characteristics of the alternans, a risk of sudden cardiac death, cardiac arrest, sudden infant death, or arrhythmias. The method can additionally include assessing, based on the determined characteristics of the alternans, the existence of ischemia or coronary artery disease.

Finally, determining whether alternans is present in the cardiac signal data segment can include determining whether T-wave alternans is present in the cardiac signal data segment and determining characteristics of the alternans can include determining characteristics of the T-wave alternans. Determining whether alternans is present in the cardiac signal data segment can include determining whether ST segment alternans is present in the cardiac signal data segment and determining characteristics of the alternans can include determining characteristics of the ST segment alternans. Determining whether alternans is present in the cardiac signal data segment can include determining whether QRS complex alternans is present in the cardiac signal data segment and determining characteristics of the alternans can include determining characteristics of the QRS complex alternans.

In other implementations, some aspects include a computer-readable medium encoded with a computer program comprising instructions that, when executed, operate to cause a computer to perform operations. The operations include accessing cardiac signal data of heart beats measured with an ambulatory electrocardiography device and segmenting the cardiac signal data into cardiac signal data segments, each cardiac signal data segment including cardiac signal data of sequential heart beats. The operations also include determining, for each of multiple cardiac signal data segments, whether alternans is present in the cardiac signal data segment and determining alternans characteristics by analyzing cardiac signal data segments for which alternans is determined to be present or characteristics of cardiac signal data segments for which alternans is determined to be present.

In other implementations, some aspects include a system including sensors configured to measure electrical activity of heart beats, an amplifier configured to amplify the electrical activity measured by the sensors, an analog to digital converter configured to convert the electrical activity measured by the sensors to digital signals, and a processor. The processor is configured to receive the digital signals and generate cardiac signal data segments, with each cardiac signal data segment including cardiac signal data of sequential heart beats. The processor is also configured to determine, for each of multiple cardiac signal data segments, a characteristic pertaining to the cardiac signal data segment and store, for each of the multiple cardiac signal data segments, the generated cardiac signal data segment along with the determined characteristic pertaining to the cardiac signal data segment.

This and other implementations can optionally include one or more of the following features, which also may optionally be in any combination. For example, the system can include a non-volatile storage unit configured to interface with multiple devices. To store the generated cardiac signal data segments along with the determined characteristics pertaining to the cardiac signal data segments, the processor can be configured to store the cardiac signal data segments and the characteristics pertaining to the cardiac signal data segments on the non-volatile storage unit. The non-volatile storage unit can be a flash drive. The system can also include a display. The processor can be configured to generate display information based upon one or more determined characteristics pertaining to one or more generated cardiac signal data segments and the display can be configured to render the display information generated by the processor.

Also, to determine the characteristic pertaining to the cardiac signal data segment, the processor can be configured to determine a heart rate pertaining to the cardiac signal data segment and to store the cardiac signal data segment along with the characteristic pertaining to the cardiac signal data segment, the processor can be configured to store the cardiac signal data segment along with the determined heart rate pertaining to cardiac signal data segment. To determine the characteristic pertaining to the cardiac signal data segment, the processor can be configured to determine whether alternans is present in the cardiac signal data segment and to store the cardiac signal data segment along with the characteristic pertaining to the cardiac signal data segment, the processor can be configured to store the cardiac signal data segment along with an indication of the determined presence of alternans in the cardiac signal data segment.

Further, the processor can be configured to determine an onset heart rate of alternans for the cardiac signal data segments and store an indication of the determined onset heart rate for the cardiac signal data segments. The processor can be configured to determine a maximum heart rate below which alternans is not present for the cardiac signal data segments and store an indication of the determined maximum heart rate below which alternans is not present for the cardiac signal data segments. The processor can be configured to determine presence or absence of alternans sustained for a period of time for the cardiac signal data segments and store an indication of the determined presence or absence of alternans sustained for a period of time for the cardiac signal data segments. The processor can be configured to generate the cardiac signal data segments such that the sequential order of the heart beats as measured by the sensors is maintained within the cardiac signal data segments. The processor can be configured to generate the cardiac signal data segments such that the cardiac signal data in each cardiac signal data segment partially overlaps the cardiac signal data of another cardiac signal data segment.

Finally, the processor can be configured to generate the cardiac signal data segments such that each cardiac signal data segment includes cardiac signal data of at least one measured heart beat which is also included in another cardiac signal data segment. The processor can be configured to generate the cardiac signal data segments such that the cardiac signal data of each measured heart beat is included in at least one cardiac signal data segment along with the cardiac signal data of the sequentially previous measured heart beat and the cardiac signal data of the sequentially following measured heart beat.

The details of one or more implementations are set forth in the accompanying drawings and the description below. Other features will be apparent from the description and drawings, and from the claims.

### DESCRIPTION OF DRAWINGS

FIG. 1 is an example of an electrical waveform of a heart beat measured to produce cardiac signal data.

FIG. 2 is an illustration of a patient using an ambulatory electrocardiography device.

FIG. 3 is a schematic of an ambulatory electrocardiography device.

FIG. 4 is a block diagram of a process to detect alternans using an ambulatory electrocardiography device.

FIG. 5 is a diagram of a heart rate profile of cardiac signal data stored by an ambulatory electrocardiography device.

FIG. 6 is a diagram of a heart rate profile of segmented cardiac signal data generated from the cardiac signal data of FIG. 5.

FIG. 7 is a diagram of sorted cardiac signal data generated from the segmented cardiac signal data of FIG. 6.

FIG. 8 is a schematic of a computer system configured to carry out the process of FIG. 4.

### DETAILED DESCRIPTION

Alternans is a pattern of variation of an electrocardiographic complex. Specifically, alternans can represent an every-other-beat pattern of variation in the electrical activity of the heart resulting in an every-other-beat variation in the shape or amplitude of electrocardiographic waveforms. Alternans can be used to predict susceptibility to sudden cardiac death, sudden cardiac arrest and life threatening ventricular arrhythmias. Also, alternans can be used to detect cardiac ischemia or coronary artery disease. Alternans is generally more often detected when a patient's heart rate is elevated, such as between 100-120 beats per minute (BPM). Detection and analysis of alternans in a patient's heartbeat allows for formulation of a treatment plan including preemptive measures, such as medication or use of an internal cardioverter/defibrillator to help prevent future medical problems.

Alternans is generally measured as small voltage changes, such as a few microvolts, using an electrocardiogram (ECG) produced by an electrocardiography device operated by a doctor or technician. The ECG is a measurement of heart beats and can be produced in a controlled setting, such as a hospital or doctor's office to obtain cardiac signal data at a desired heart rate while controlling for noise. This can involve techniques such as placing the patient on a treadmill to intentionally elevate the heart rate and using an impedance measurement to factor out signal noise. Measuring alternans using an electrocardiography device for an extended period of time is often not practical, as the patient is generally confined to the location of the ECG device and the measurements require the ongoing involvement of the technician or doctor.

An ambulatory electrocardiography device (AED) is a portable electrocardiography device configured to be worn on a patient's person. The patient wears the AED outside of the hospital or doctor's office without having their mobility significantly limited. An AED may also be an implantable device. The AED measures and stores cardiac signals for an extended period of time (e.g., 24 hours). AEDs may differ from stationary instrumentation generally used in medical facilities. For example, AEDs may not include an impedance measurement or respiration measurement and may utilize fewer recording electrodes. AEDs may record electrical signals throughout various patient activity and in various environments. Consequently, the cardiac signal data produced by an AED can be of a wide range of heart rates and can have higher levels of noise. To compensate for these and/or other issues, the processing techniques used to analyze the AED's cardiac signal data to detect alternans can be different than that used to analyze the ECG of an electrocardiography device.

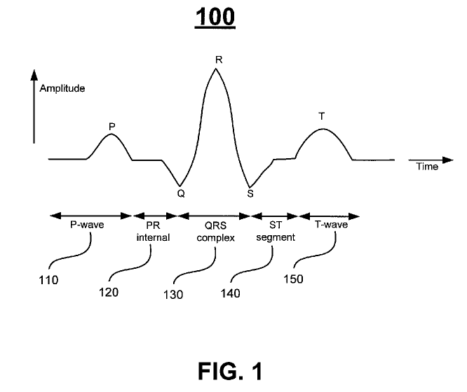

FIG. 1 is an example of a waveform 100 of a heart beat measured by an electrocardiography device. In particular, the waveform 100 is a measurement of a voltage between two electrodes placed on the body surface. The waveform 100 corresponds to a single heart beat. Various portions of the waveform 100 represent electrical activity in various structures of the heart. The P-wave 110 of the waveform 100 appears at initiation of the beat and corresponds to electrical activation of the atria of the heart. The PR interval 120 corresponds to the time between the end of the P-wave 110 and the onset of the QRS complex 130. There is normally no measurable electrical activity during the PR interval and this interval is often used to set the zero baseline of the recording. The QRS complex 130 corresponds to the electrical activation of the ventricles. The ST segment 140 represents the period between the end of the QRS complex and the onset of the T-wave 150 and corresponds to the portion of time during which the ventricles are activated (depolarized). In normal individuals the ST segment tends to be relatively flat or slightly up-sloping and is approximately at the zero baseline. However, the ST segment can be shifted up or down or have a nonzero slope in patients with myocardial disease. The T-wave 150 reflects the electrical recovery of the ventricles.

Alternans can be an every other beat pattern of variation in the shape or amplitude of part of the waveform 100. For example, T-wave alternans is an every-other-beat pattern of variation in the shape or amplitude of the T-wave. The presence of T-wave alternans can indicate electrical instability of the ventricles. Clinically significant T-wave alternans can be measured as only a few microvolts and can be masked by other temporal patterns of beat-to-beat variability in the waveform 100. For example, skeletal muscle activity, electrode and cable motion, ambient electromagnetic activity, and device amplifiers all can introduce signal noise of a larger amplitude than that of the T-wave alternans. In the following description, T-wave alternans is generally referred to (rather than alternans of other portions of the waveform 100) for simplicity of understanding, though alternans of other portions of the waveform 100 (for example, ST segment alternans and QRS complex alternans) may be similarly measured and analyzed using the techniques described.

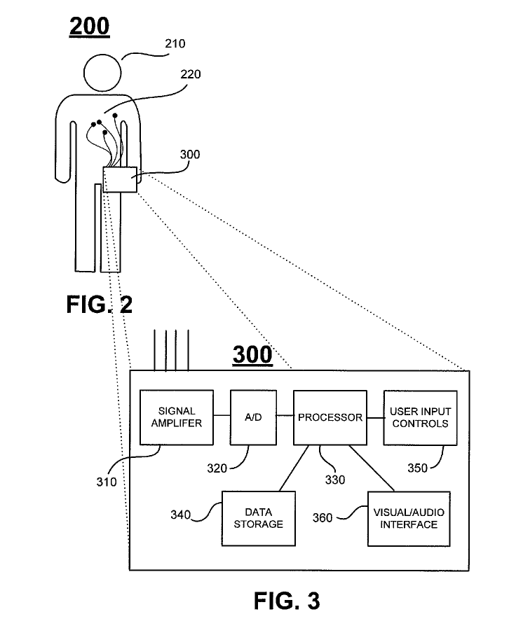

FIG. 2 is an illustration 200 of a patient 210 using an AED 300 to generate cardiac signal data and FIG. 3. is an exemplary schematic 300 of the AED 300. The cardiac signal data is processed to detect alternans in the cardiac activity of the patient 210. Also, the AED 300 enables the monitoring of the patient 210 to occur over an extended period of time and during normal physical activities.

Multiple electrodes 220 of the AED 300 are taped or otherwise attached to the chest of the patient 210 at particular locations of the patient's body to detect electrical activity from various sources. AEDs 300 generally use fewer electrodes 220 (e.g., three to eight) than electrocardiography devices (e.g., ten) to enhance device mobility. The AED 300 is generally worn at or around the patient's waist. This configuration enables the patient 210 to walk and otherwise be mobile while the AED 300 measures heart beats and records cardiac signals using the electrodes 220.

As shown, the AED 300 includes a signal amplifier 310, an analog to digital converter 320, a processor 330, and data storage 340. The AED 300 can optionally include user input controls 350 and a visual or audio interface 360. These features of the AED 300 are exemplary, the AED can include different or additional features.

The signal amplifier 310 receives the cardiac signals measured from the electrodes 220 and amplifies them to produce amplified signal channels for processing. While an ECG device typically can have 12 channels, AEDs generally have less, such as three or four channels. The signal amplifier 310 can be an instrumentation amplifier or another differential amplifier.

The amplified channels of the cardiac signals are digitized by the analog to digital converter 320 and then sent to the processor 330. Although not shown, one or more of the measured signals may be signals used to determine and adjust for noise rather than cardiac signals. For example, the AED 300 may include a signal line to measure respiration and a signal line to measure impedance. These techniques are described in more detail in U.S. Pat. No. 5,713,367, entitled “Measuring and accessing cardiac electrical stability,” the contents of which are incorporated herein by reference.

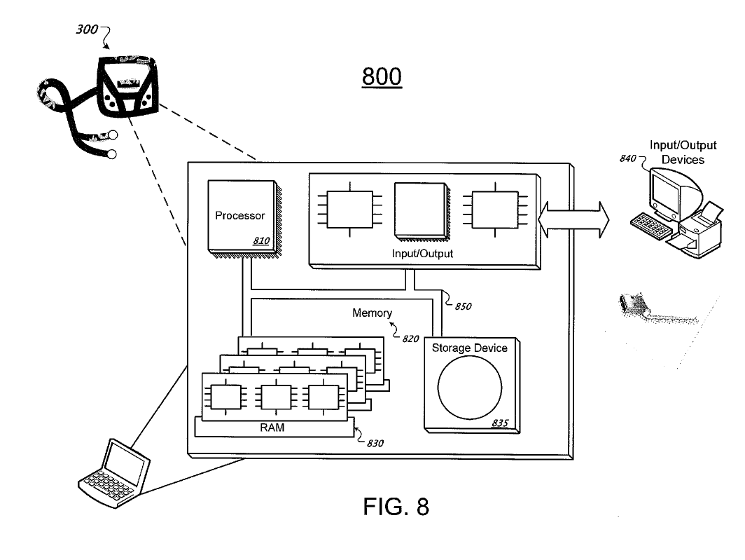

In a less complex AED 300, the processor 330 generally is directed only to the storage of the digitized channels as cardiac signal data on the data storage 340 and its communication to another device. The data storage 340 can be a tangible computer-readable storage medium, such as, for example, a flash drive or a computer hard disk. The data storage 340 itself can be removable from the AED 300 to enable uploading of the cardiac signal data to a computer or other device. Also, the processor 330 can include a data communication port (e.g., a universal serial bus or Ethernet interface) to enable the AED 300 to interface with a computer to upload, display, or process cardiac signal data. Additional computer hardware and functionality which can be included in the AED 300 is included in the description of FIG. 8 below.

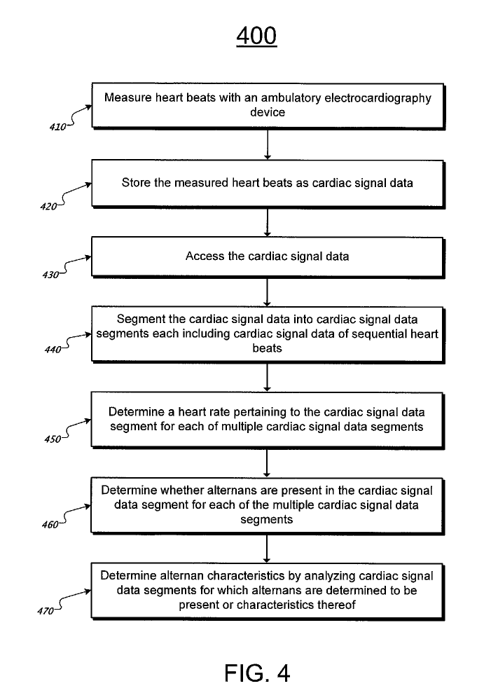

In a more complex AED 300, however, the processor 330 may itself process the cardiac signal data and may serve as an alternative to processing the cardiac signal data on a computer after uploading. Processing of the cardiac signal data is described in more detail in the description of the process 400 of FIG. 4. The processor 330 also can include user input controls 350 and a visual or audio interface 360 to enable additional functionality to better enable the measurement of cardiac signals useful in detecting alternans. For example, T-wave alternans is more often detected at heart rates of between 100 and 120 BPM. The user input controls 350 and the visual or audio interface 360 can be used to communicate whether additional signal data is needed from such an accelerated heart rate. The patient 210 can use this information to determine whether he/she needs to spend time in a physically active state to facilitate the desired measurement of cardiac signals. Further information of how AEDs can be used is described below, after the description of the process 400 of FIG. 4.

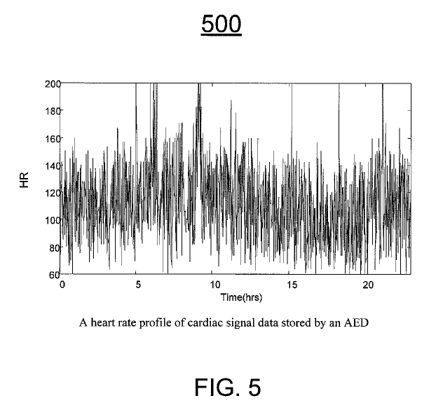

FIG. 4 is a block diagram of an example of a process 400 to detect alternans using an AED. The process 400 is described with respect to the features of FIGS. 2 and 3, though different AEDs or different features may be used. Also, the below description of the process 400 refers to FIGS. 5-7, which are exemplary diagrams which can be representative of cardiac signal data analyzed during the process 400.

A patient 210 wears the AED 300 with the electrodes 220 taped to parts of his/her chest. The patient's heart beats generate cardiac signals as voltages in the electrodes 220. The heart beats are measured with the AED 300 (410). Specifically, the AED 300 amplifies and digitizes the voltages from the electrodes 220 to enable digital signal processing by the processor 330 of the AED 300.

The AED 300 stores the measured heart beats as cardiac signal data (420). Many AEDs store the cardiac signal data in transferable memory (e.g., a flash drive) to enable the data to be further processed elsewhere.

The cardiac signal data of heart beats measured with the AED 300 can be accessed by the AED 300 or a separate device (430). By using the separate device in further processing, the AED 300 can be of minimal size and complexity. Nevertheless, a more advanced AED 300 with additional processing power and programming can implement the further processing discussed below without the use of a separate device.

FIG. 5 is a diagram 500 of an example of a heart rate profile of cardiac signal data stored by the AED 300. The diagram 500 shows cardiac signal data produced from the cardiac signals measured by the AED 300 during a 24 hour period. The cardiac signal data is presented as heart rate as a function of time. The diagram 500 illustrates the challenge of using cardiac signal data produced by the AED 300 to detect alternans.

As described above, alternans can be a beat-to-beat variation on an every-other-beat basis of portions of the waveform 100 of a measured cardiac signal. For example, T-wave alternans can be microvolt-level variation in the shape or amplitude of the T-wave from one beat to the next. Optimally, to detect T-wave alternans, the cardiac signal data is both at a heart rate of 100 to 120 BPM and is maintained at that level long enough to repeatedly analyze the beat-to-beat variation. However, the cardiac signal data of the diagram 500 is not consistently at the desired heart rate and is not maintained at a given level. Although there are instances where the heart rate is between 100 and 120 BPM, these instances are scattered and not ideal for the detection of alternans.

In one implementation, the cardiac signal data stored by the AED 300 is processed to convert the scattered cardiac data of the diagram 500 into more useful data organized by associated heart rates. Simply sorting cardiac signal data by heart rate for each beat can foreclose the detection of variations between consecutive beats. Therefore, to preserve the beat-to-beat nature of the cardiac signal data, the processing can involve segmenting data into groups of adjacent beats, determining a heart rate for the segments, and sorting the segments by the heart rate prior to processing to detect and analyze alternans.

The cardiac signal data is segmented into cardiac signal data segments (440). Each cardiac signal data segment includes data associated with multiple consecutive heartbeats. In one implementation, the segments are of 128 beats, but other segment sizes can be used. The segments can overlap beats so as to ensure the temporal relationship of beats is not lost. For example, the first 248 beats of cardiac signal data can be segmented into a first segment of beats 1 to 128 and a second segment of beats 120 to 248, leaving beats 120-128 included in both segments. Therefore beat-to-beat variations in beats 120-128 can be compared to beats occurring just prior to beats 120-128 as well as to beats occurring just after beats 120-128.

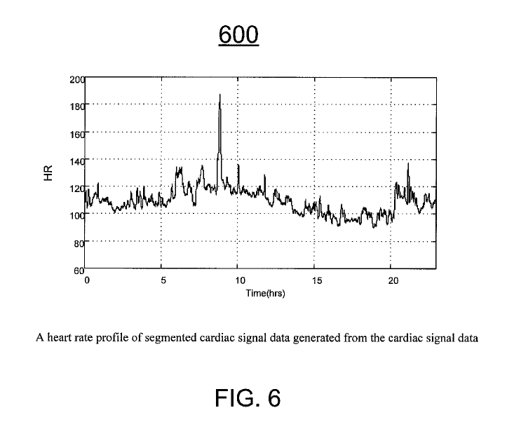

A heart rate pertaining to the cardiac signal data segment is determined for each of multiple cardiac signal data segments (450). In particular, a heart rate is separately calculated for each of the cardiac signal data segments. The heart rate can be based on a simple averaging of the duration of each of the heart beats of a cardiac signal data segment, such as an average duration of each PQRST complex. FIG. 6 is a diagram 600 of an example of a heart rate profile of segmented cardiac signal data generated from the cardiac signal data of FIG. 5. The diagram 600 shows the segmented cardiac signal data as heart rate as a function of time. Notably, the heart rate of the segmented cardiac signal data in the diagram 600 fluctuates less dramatically than the heart rate of the cardiac signal data of individual heart beats as shown in the diagram 500.

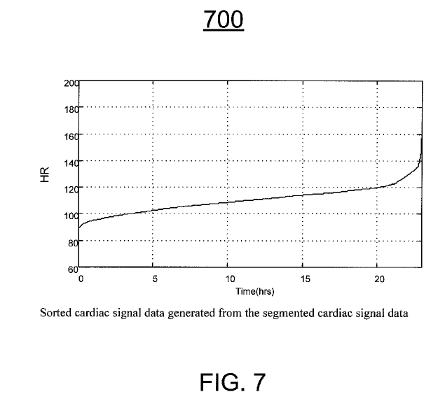

In some implementations, the cardiac signal data segments are sorted into an order from the lowest determined heart rate to the highest determined heart rate. FIG. 7 is a diagram 700 of an example of sorted cardiac signal data segments generated from the segmented cardiac signal data of FIG. 6. The diagram 700 shows the distribution of heart rates for the segments after the segments have been ordered from the lowest determined heart rate to the highest determined heart rate. Although this exemplary distribution shows that the majority of cardiac signal data segments fall within the desired heart rate of 100 to 120 BPM, other distributions from other patients can commonly have only a small fraction of the cardiac signal data segments within the desired heart rate.

Whether alternans is present in the cardiac signal data segment is determined for each of the multiple cardiac signal data segments (or each of the cardiac signal data segments corresponding to suitable heart rates) (460). In particular, each of the cardiac signal data segments is separately processed to detect alternans. Therefore, each of the cardiac signal data segments can have a unique determination of the presence and/or characteristics of alternans.

Many implementations use spectral or analytic approaches to determine the presence of alternans in the cardiac signal data segments. These approaches are described in detail in U.S. Pat. No. 7,197,358, entitled “Identifying Infants at Risk for Sudden Infant Death Syndrome,” the contents of which are incorporated herein by reference. In the example above, where the first 248 beats of cardiac signal data are segmented into a first segment of beats 1 to 128 and a second segment of beats 120 to 248, the first segment is analyzed using the spectral or analytical approach to determine a first result and the second segment is then analyzed using the spectral or analytical approach to determine a second result.

The analysis of the cardiac signal data segments can also include processing dependent upon the determined heart rate or other characteristics of the cardiac signal data segments. In some implementations, cardiac signal data segments outside of a given range may be discarded or removed from further consideration. For example, cardiac signal data segments with determined heart rates below 100 BPM or above 120 BPM may be excluded from further processing. In other implementations, processing is conducted differently based upon the determined heart rate. For example, cardiac signal data segments with determined heart rates below 100 BPM may undergo a first type of further processing, whereas cardiac signal data segments with determined heart rates above 100 BPM may undergo a second type of further processing.

Turning to the spectral approach, this approach uses measurements from time synchronized points of consecutive T waves. For a cardiac signal data segment, a time series is created by measuring, for each of the heart beats, the T-wave level at a fixed location relating to the QRS complex of the waveform. This process is repeated to create a time series for each location in the T-wave of the heart beats in the cardiac signal data segment. A frequency spectrum is then generated for each time series, and the spectra are averaged to form a composite T-wave alternans spectrum.

Since the T-waves are sampled once per beat for each time series, the spectral value at the Nyquist frequency, i.e. 0.5 cycles per beat, indicates the level of beat-to-beat alternation in the T-wave waveform. The alternans power is calculated from the composite T-wave alternans spectrum and statistically compared to the noise power to discriminate the beat-to-beat T-wave variation due to abnormal electrical activity of the heart from the random variation due to background noise. Alternans may be considered to be significant if the alternans exceed noise by a threshold amount, such as at least three times the standard deviation of the noise in a given noise reference band.

One example of how processing can be conducted differently based upon the determined heart rate is using a different threshold for determining whether the alternans is significant for cardiac signal data segments of different heart rate ranges. For example, alternans of cardiac signal data segments with determined heart rates below 100 BPM may be considered significant if the alternans is at least double the standard deviation of the noise in the noise reference band, whereas alternans of cardiac signal data segments with determined heart rates above 100 BPM may be considered significant if the alternans is at least triple the standard deviation of the noise in the noise reference band.

Turning to the analytic approach, this approach can be used to minimize the presence of noise or artifacts. First, the cardiac signal data segment is low-pass filtered. In one implementation, the low pass filter is a 5^{th} order Butterworth filter with a zero phase configuration. The cardiac signal data segment is transferred to the frequency domain using a fast Fourier transform (FFT). In the frequency domain, the portions of the frequency spectrum corresponding to negative frequencies are removed and all positive, non-zero components of the frequency spectrum are doubled to compensate. An inverse fast Fourier transform (IFFT) is then performed on the modified frequency spectrum to produce an analytical data segment in the time domain. Next, the analytical data segment is referenced to an analytical version of Wilson's central terminal (WCT), an ECG reference value. The analytical version of WCT is generated from the standard WCT using the procedures described in U.S. Pat. No. 7,197,358, title “Identifying infants at risk for sudden infant death syndrome” and U.S. Pat. No. 5,704,365, titled “Using Related Signals to Reduce ECG Noise,” the contents of both are incorporated herein by reference. The analytical data segment is referenced to the analytical version of WCT by determining the difference between the two. The referenced analytical data segment then is processed.

If the data from the AED 300 includes signals used to determine and adjust for noise (e.g., signals related to respiration and impedance), the time series can be processed to reduce noise such as that resulting from baseline wander. Techniques for processing the time series are described in more detail in U.S. Pat. No. 5,704,365, titled “Using Related Signals to Reduce ECG Noise,” the contents of which are incorporated herein by reference.

Next, characteristics of the alternans are determined by analyzing cardiac signal data segments for which alternans is determined to be present or characteristics of cardiac signal data segments for which alternans is determined to be present (470). In particular, the occurrences of alternans in cardiac signal data segments can be compared to the context of the occurrences to determine further information. The context of the occurrence can include the heart rate pertaining to the cardiac data segment, the temporal position of a cardiac signal data segment with alternans present relative to other cardiac signal data segments, the consecutive duration of cardiac signal data segments with alternans, the time or heart rate of the first cardiac signal data segment with alternans present, the type of activity the patient was involved in at the time of the segment, the presence of other ECG abnormalities detected in the data segment (for example, changes in the ST segment which may be indicative of ischemia), and other considerations.

This analysis can determine alternans characteristics, such as the alternans onset heart rate, the maximum heart rate below which alternans is not detected, the duration of alternans, the distribution of alternans, and other characteristics. Based on the alternans characteristics, a probability of risk or diagnosis for medical problems, such as a ventricular tachyarrhythmic event, can be determined. In some implementations, the probability of risk is determined by comparing the alternans onset heart rate and the distribution of heart rates with alternans. Further information about the analysis and classification of measured alternans can be found at U.S. application Ser. No. 6,453,191 entitled “Automated Interpretation of T-wave Alternans Results,” the contents of which are incorporated herein by reference.

Information of the process 400 can be made accessible by, for example, graphically displaying diagrams of data produced in the process 400 or the results of the determined characteristics of the alternans, probability of risk, or diagnosis. The information of the process 400 can also be made accessible by storing diagrams of data produced in the process 400 or the results of the determined alternans characteristics, probability of risk, or diagnosis in machine readable format.

As noted above, the process 400 can be carried out using the AED 300 to measure cardiac signals and store cardiac signal data, and using a separate computer to conduct further processing. More advanced AEDs can be programmed to themselves carry out the processing of the process 400. In some implementations, the AED 300 itself segments the data, determines the heart rate of segments, determines the presence of alternans in the segments, and/or determines the alternans characteristics using the processor 330 of the AED 300 concurrent with the measuring of cardiac signal data. In these implementations, the AED 300 may store and access the cardiac signal data, generated cardiac signal data segments, determined characteristics, or any other information discussed above in and from volatile memory along with or instead of non-volatile memory to enable further processing to be carried out concurrently with measurement rather than after measurement. For example, in some implementations, the segmented first and second cardiac data is stored in the data storage 340 and is accessed by another device. Thereafter, the other device completes the process 400.

The AED 300 can store all cardiac signal data segments along with and associated with the determined characteristics if any (e.g., heart rate, presence of alternans, or onset heart rate of alternans) in the data storage 340. Also, the AED 300 can store only cardiac signal data segments in the data storage 340 if a relevant characteristic is determined (e.g., only if the segment includes alternans or is within a desired heart rate). Also, live information of detected alternans or characteristics of detected alternans (e.g., duration or onset heart rate) can be generated by the processor 330 and displayed to the patient 210 using the user input controls 350 and the visual or audio interface 360. If alternans is detected, the patient 210 can be informed as the detection occurs along with information of characteristics of the alternans, such as, the onset heart rate.

FIG. 8 is a schematic of an example of a computer system 800 configured to carry out the process 400 of FIG. 4. While the computer system 800 is generally described as a separate device from the AED 300 of FIG. 3, the description of the computer system 800 can also apply to the hardware and functioning of the AED 300.

The computer system 800 includes a processor 810, memory 820, and an input/output device 840. The components 810, 820, and 840 are interconnected using a system bus 850. The processor 810 is capable of processing instructions for execution within the computer system 800. In one implementation, the processor 810 is a single-threaded processor. In another implementation, the processor 810 is a multi-threaded processor. The processor 810 is capable of processing instructions stored in the memory 820 to display graphical information for a user interface on the input/output device 840.

The memory 820 stores information within the computer system 800 and includes volatile memory 830 and non-volatile memory 835 and can be a computer-readable medium tangibly embodying instructions. The volatile memory 830 can include random access memory (RAM) and semiconductor memory devices (e.g., flip-flops or registers). The non-volatile memory 835 is capable of providing mass storage for the computer system 800. In various implementations, the non-volatile memory 835 can be a floppy disk device, a hard disk device, an optical disk device, or a tape device. Also, the non-volatile memory 835 can include, or be operatively coupled to communicate with, one or more mass storage devices for storing data files; such devices include magnetic disks, such as internal hard disks and removable disks, magneto-optical disks, optical disks, EPROM, EEPROM, flash memory devices, and CD-ROM, DVD-ROM, or Blu-ray™ disks.

The input/output device 840 provides input/output operations for the computer system 800. In one implementation, the input/output device 840 includes a keyboard and/or pointing device. In another implementation, the input/output device 840 includes a display unit for displaying graphical user interfaces. The input/output device 840 can include communications input/output operations. For example, the input/output device 840 can include a port for connection flash drives or other memory devices through a universal serial bus or other connection. Also, the input/output device 840 can include an Ethernet port for communication with other devices.

The features and processing described above can be implemented in a computer program product tangibly embodied in an information carrier, e.g., in a computer-readable medium encoded with a computer program product or in a machine-readable storage device for execution by a programmable processor; and features of the methods may be performed by a programmable processor executing a program of instructions to perform functions of the described implementations by operating on input data and generating output.

The described features and processing may be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that may be used, directly or indirectly, in a computer to perform a certain activity or bring about a certain result. A computer program may be written in any form of programming language, including compiled or interpreted languages, and it may be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. The processor and the memory may be supplemented by, or incorporated in, ASICs (application-specific integrated circuits).

To provide for interaction with a user, the features may be implemented on a computer having a display device such as a CRT (cathode ray tube) or LCD (liquid crystal display) monitor for displaying information to the user and a keyboard and a pointing device such as a mouse or a trackball by which the user may provide input to the computer.

The components of the system may be connected by any form or medium of digital data communication such as a communication network. Examples of communication networks include, e.g., a LAN, a WAN, and the computers and networks forming the Internet.

A number of implementations have been described. Nevertheless, it will be understood that various modifications may be made without departing from the spirit and scope of the claims. For example, the flow diagram depicted in the figures does not require the particular order shown, or sequential order, to achieve desirable results. In addition, other features may be provided, or features may be eliminated, from the described block diagram, and other components may be added to, or removed from, the described devices. Accordingly, other implementations are within the scope of the following claims.

What is claimed is:

1. A method comprising:
accessing cardiac signal data of heart beats measured with an ambulatory electrocardiography device;
segmenting the cardiac signal data into cardiac signal data segments, each cardiac signal data segment including cardiac signal data of sequential heart beats;
determining, for each of multiple cardiac signal data segments, whether alternans is present in the cardiac signal data segment; and
determining characteristics of the alternans by analyzing cardiac signal data segments for which alternans is determined to be present or characteristics of cardiac signal data segments for which alternans is determined to be present.

2. The method of claim 1 further comprising determining, for one or more of the cardiac signal data segments, a heart rate pertaining to the cardiac signal data segment.

3. The method of claim 2 wherein determining a heart rate pertaining to the cardiac signal data segment includes determining an average heart rate of the heart beats of the cardiac signal data in the cardiac signal data segment.

4. The method of claim 2 wherein determining characteristics of the alternans includes analyzing the determined heart rates pertaining to the cardiac signal data segments for which alternans is determined to be present.

5. The method of claim 4 wherein analyzing the determined heart rates pertaining to the cardiac signal data segments for which alternans is determined to be present includes determining an onset heart rate of alternans for the cardiac signal data segments.

6. The method of claim 4 wherein analyzing the determined heart rates pertaining to the cardiac signal data segments for which alternans is determined to be present includes determining a maximum heart rate below which alternans is not present for the cardiac signal data segments.

7. The method of claim 1 wherein determining characteristics of the alternans includes determining a presence or absence of alternans sustained for a period of time for the cardiac signal data segments.

8. The method of claim 1 further comprising:
measuring the heart beats with the ambulatory electrocardiography device;
generating the cardiac signal data with a processor of the ambulatory electrocardiography device; and
storing the cardiac signal data in a storage unit of the ambulatory electrocardiography device.

9. The method of claim 8 wherein accessing the cardiac signal data of heart beats measured with the ambulatory electrocardiography device includes accessing the stored cardiac signal data in the storage unit with a device other than the ambulatory electrocardiography device.

10. The method of claim 1 wherein segmenting the cardiac signal data includes segmenting the cardiac signal data such that the sequential order of the heart beats as measured by the ambulatory electrocardiography device is maintained within the cardiac signal data segments.

11. The method of claim 1 wherein segmenting the cardiac signal data includes segmenting the cardiac signal data such that the cardiac signal data in each cardiac signal data segment partially overlaps the cardiac signal data of another cardiac signal data segment.

12. The method of claim 11 wherein segmenting the cardiac signal data includes segmenting the cardiac signal data such that each cardiac signal data segment includes cardiac signal data of at least one measured heart beat which is also included in another cardiac signal data segment.

13. The method of claim 11 wherein segmenting the cardiac signal data includes segmenting the cardiac signal data such that the cardiac signal data of each measured heart beat is included in at least one cardiac signal data segment along with the cardiac signal data of the sequentially previous measured heart beat and the cardiac signal data of the sequentially following measured heart beat.

14. The method of claim 1 wherein determining, for each of multiple cardiac signal data segments, whether alternans is present in the cardiac signal data segment includes making a separate determination of whether alternans is present for each of the cardiac signal data segments.

15. The method of claim 14 wherein making a separate determination of whether alternans is present for each of the cardiac signal data segments includes conducting spectral or analytic processing separately on each cardiac signal data segment.

16. The method of claim 14 further comprising determining that alternans is present in a first cardiac signal data segment and are not present in a second cardiac signal data segment.

17. The method of claim 1 further comprising rendering information of the determined characteristics of the alternans.

18. The method of claim 1 wherein accessing cardiac signal data of heart beats measured with an ambulatory electrocardiography device includes accessing cardiac signal data of heart beats measured with an implanted device.

19. The method of claim 1 further comprising determining the existence of alterations in the electrocardiographic waveforms which persist over multiple beats.

20. The method of claim 19 wherein determining the existence of alterations in the electrocardiographic waveforms includes determining ST segment changes.

21. The method of claim 1 further comprising:
determining physical activities occurring during measurement of cardiac signal data with the ambulatory electrocardiography device; and
analyzing the determined characteristics of the alternans based on the determined physical activities.

22. The method of claim 1 further comprising assessing, based at least in part on the determined alternans characteristics, a risk of sudden cardiac death, cardiac arrest, sudden infant death, or arrhythmias.

23. The method of claim 1 further comprising assessing the existence of ischemia or coronary artery disease.

24. The method of claim 1 wherein:
determining whether alternans is present in the cardiac signal data segment includes determining whether T-wave alternans is present in the cardiac signal data segment; and
determining characteristics of the alternans includes determining characteristics of the T-wave alternans.

25. The method of claim 1 wherein:
determining whether alternans is present in the cardiac signal data segment includes determining whether ST segment alternans is present in the cardiac signal data segment; and
determining characteristics of the alternans includes determining characteristics of the ST segment alternans.

26. The method of claim 1 wherein:
determining whether alternans is present in the cardiac signal data segment includes determining whether QRS complex alternans is present in the cardiac signal data segment; and
determining characteristics of the alternans includes determining characteristics of the QRS complex alternans.

27. A computer-readable medium encoded with a computer program comprising instructions that, when executed, operate to cause a computer to perform operations comprising:
accessing cardiac signal data of heart beats measured with an ambulatory electrocardiography device;
segmenting the cardiac signal data into cardiac signal data segments, each cardiac signal data segment including cardiac signal data of sequential heart beats;
determining, for each of multiple cardiac signal data segments, whether alternans is present in the cardiac signal data segment; and
determining characteristics of the alternans by analyzing cardiac signal data segments for which alternans is determined to be present or characteristics of cardiac signal data segments for which alternans is determined to be present.

28. A system comprising:
sensors configured to measure electrical activity of heart beats;
an amplifier configured to amplify the electrical activity measured by the sensors;
an analog to digital converter configured to convert the electrical activity measured by the sensors to digital signals; and
a processor configured to:
receive the digital signals,
generate cardiac signal data segments, each cardiac signal data segment including cardiac signal data of sequential heart beats,
determine, for each of multiple cardiac signal data segments, a characteristic pertaining to the cardiac signal data segment, and
store, for each of the multiple cardiac signal data segments, the generated cardiac signal data segment along with the determined characteristic pertaining to the cardiac signal data segment.

29. The system of claim 28 further comprising a non-volatile storage unit configured to interface with multiple devices, wherein:
to store the generated cardiac signal data segments along with the determined characteristics pertaining to the cardiac signal data segments, the processor is configured to store the cardiac signal data segments and the characteristics pertaining to the cardiac signal data segments on the non-volatile storage unit.

30. The system of claim 29 wherein the non-volatile storage unit is a flash drive.

31. The system of claim 28 further comprising a display, wherein:
the processor is configured to generate display information based upon one or more determined characteristics pertaining to one or more generated cardiac signal data segments; and
the display is configured to render the display information generated by the processor.

32. The system of claim 28 wherein:
to determine the characteristic pertaining to the cardiac signal data segment, the processor is configured to determine a heart rate pertaining to the cardiac signal data segment; and
to store the cardiac signal data segment along with the characteristic pertaining to the cardiac signal data segment, the processor is configured to store the cardiac signal data segment along with the determined heart rate pertaining to cardiac signal data segment.

33. The system of claim 28 wherein:
to determine the characteristic pertaining to the cardiac signal data segment, the processor is configured to determine whether alternans is present in the cardiac signal data segment; and
to store the cardiac signal data segment along with the characteristic pertaining to the cardiac signal data segment, the processor is configured to store the cardiac signal data segment along with an indication of the determined presence of alternans in the cardiac signal data segment.

34. The system of claim 33 wherein the processor is configured to:
determine an onset heart rate of alternans for the cardiac signal data segments; and
store an indication of the determined onset heart rate for the cardiac signal data segments.

35. The system of claim 33 wherein the processor is configured to:
determine a maximum heart rate below which alternans is not present for the cardiac signal data segments; and
store an indication of the determined maximum heart rate below which alternans is not present for the cardiac signal data segments.

36. The system of claim 33 wherein the processor is configured to:
determine presence or absence of alternans sustained for a period of time for the cardiac signal data segments; and
store an indication of the determined presence or absence of alternans sustained for a period of time for the cardiac signal data segments.

37. The system of claim 28 wherein the processor is configured to generate the cardiac signal data segments such that the sequential order of the heart beats as measured by the sensors is maintained within the cardiac signal data segments.

38. The system of claim 28 wherein the processor is configured to generate the cardiac signal data segments such that the cardiac signal data in each cardiac signal data segment partially overlaps the cardiac signal data of another cardiac signal data segment.

39. The system of claim 38 wherein the processor is configured to generate the cardiac signal data segments such that each cardiac signal data segment includes cardiac signal data of at least one measured heart beat which is also included in another cardiac signal data segment.

40. The system of claim 28 wherein the processor is configured to generate the cardiac signal data segments such that the cardiac signal data of each measured heart beat is included in at least one cardiac signal data segment along with the cardiac signal data of the sequentially previous measured heart beat and the cardiac signal data of the sequentially following measured heart beat.
